SEQUENCE LISTING (2 sequences)

SEQ ID NO: 1 - protein, 605 aa, Artificial Sequence
  Cry1Fa
MENNIQNQCVPYNCLNNPEVEILNEERSTGRLPLDISLSLTRFLLSEFVPGVGVAFGLFDLIWGFITPSDWSLFLLQIEQLIEQRIETLERNRAITTLRGLADSYEIYIEALREWEANPNNAQLREDVRIRFANTDDALITAINNFTLTSFEIPLLSVYVQAANLHLSLLRDAVSFGQGWGLDIATVNNHYNRLINLIHRYTKHCLDTYNQGLENLRGTNTRQWARFNQFRRDLTLTVLDIVALFPNYDVRTYPIQTSSQLTREIYTSSVIEDSPVSANIPNGFNRAEFGVRPPHLMDFMNSLFVTAETVRSQTVWGGHLVSSRNTAGNRINFPSYGVFNPGGAIWIADEDPRPFYRTLSDPVFVRGGFGNPHYVLGLRGVAFQQTGTNHTRTFRNSGTIDSLDEIPPQDNSGAPWNDYSHVLNHVTFVRWPGEISGSDSWRAPMFSWTHRSATPTNTIDPERITQIPLVKAHTLQSGTTVVRGPGFTGGDILRRTSGGPFAYTIVNINGQLPQRYRARIRYASTTNLRIYVTVAGERIFAGQFNKTMDTGDPLTFQSFSYATINTAFTFPMSQSSFTVGADTFSSGNEVYIDRFELIPVTATLE

SEQ ID NO: 2 - protein, 612 aa, Artificial Sequence
  Cry1Ab
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDYAVRWYNTGLERVWGPDSRDWVRYNQFRRELTLTVLDIVALFPNYDSRRYPIRTVSQLTREIYTNPVLENFDGSFRGSAQGIERSIRSPHLMDILNSITIYTDAHRGYYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEFNNIIPSSQITQIPLTKSTNLGSGTSVVKGPGFTGGDILRRTSPGQISTLRVNITAPLSQRYRVRIRYASTTNLQFHTSIDGRPINQGNFSATMSSGSNLQSGSFRTVGFTTPFNFSNGSSVFTLSAHVFNSGNEVYIDRIEFVPAEVTFE

(19) United States
(12) Patent Application Publication
(10) Pub. No.: US 2013/0042374 A1
(43) Pub. Date: Feb. 14, 2013

(54) COMBINED USE OF Cry1Fa AND Cry1Ab PROTEINS FOR CONTROL OF 1Ab-RESISTANT SUGARCANE BORER AND FOR INSECT RESISTANCE MANAGEMENT IN SUGARCANE

(71) Applicants: Thomas Meade, Zionsville, IN (US); Kenneth Narva, Zionsville, IN (US); Nicholas P. Storer, Kensington, MD (US); Joel J. Sheets, Zionsville, IN (US); Stephanie L. Burton, Indianapolis, IN (US)

(72) Inventors: Thomas Meade, Zionsville, IN (US); Kenneth Narva, Zionsville, IN (US); Nicholas P. Storer, Kensington, MD (US); Joel J. Sheets, Zionsville, IN (US); Stephanie L. Burton, Indianapolis, IN (US)

(73) Assignee: DOW AGROSCIENCES LLC, Indianapolis, IN (US)

(21) Appl. No.: 13/516,619

(22) PCT Filed: Dec. 16, 2010
(86) PCT No.: PCT/US10/60825

§ 371 Date: Oct. 30, 2012

Related U.S. Application Data
(60) Provisional application No. 61/284,289, filed on Dec. 16, 2009.

(51) Int. Cl. A01H 5/00

(52) U.S. Cl. 800/302

(57) ABSTRACT

The subject invention includes methods and sugarcane plants for controlling sugarcane borer (SCB) insects, said sugarcane plants comprising Cry1Fa and Cry1Ab core toxin containing proteins in combination to delay or prevent development of resistance by the SCB.

### BACKGROUND OF THE INVENTION

Billions of dollars are spent each year to control insect pests and additional billions are lost to the damage they inflict. Synthetic organic chemical insecticides have been the primary tools used to control insect pests but biological insecticides, such as the insecticidal proteins derived from Bacillus thuringiensis (Bt), have played an important role in some areas. The ability to produce insect-resistant plants through transformation with Bt insecticidal protein genes has revolutionized modern agriculture and heightened the importance and value of insecticidal proteins and their genes.

Several Bt proteins have been used to create the insect-resistant transgenic plants that have been successfully registered and commercialized to date. These include Cry1Ab, Cry1Ac, Cry1F and Cry3Bb in corn, Cry1Ac and Cry2Ab in cotton, and Cry3A in potato.

The commercial products expressing these proteins express a single protein except in cases where the combined insecticidal spectrum of 2 proteins is desired (e.g, Cry1Ab and Cry3Bb in corn combined to provide resistance to lepidopteran pests and rootworm, respectively) or where the independent action of the proteins makes them useful as a tool for delaying the development of resistance in susceptible insect populations (e.g., Cry1Ac and Cry2Ab in cotton combined to provide resistance management for tobacco budworm).

That is, some of the qualities of insect-resistant transgenic plants that have led to rapid and widespread adoption of this technology also give rise to the concern that pest populations will develop resistance to the insecticidal proteins produced by these plants. Several strategies have been suggested for preserving the utility of Bt-based insect resistance traits which include deploying proteins at a high dose in combination with a refuge, and alternation with, or co-deployment of, different toxins (McGaughey et al. (1998), “B.t. Resistance Management,” Nature Biotechnol. 16:144-146).

The proteins selected for use in an IRM stack need to exert their insecticidal effect independently so that resistance developed to one protein does not confer resistance to the second protein (i.e., there is not cross resistance to the proteins). If, for example, a pest population selected for resistance to “Protein A” is sensitive to “Protein B”, we would conclude that there is not cross resistance and that a combination of Protein A and Protein B would be effective in delaying resistance to Protein A alone.

In the absence of resistant insect populations, assessments can be made based on other characteristics presumed to be related to mechanism of action and cross-resistance potential. The utility of receptor-mediated binding in identifying insecticidal proteins likely to not exhibit cross resistance has been suggested (van Mellaert et al. 1999). The key predictor of lack of cross resistance inherent in this approach is that the insecticidal proteins do not compete for receptors in a sensitive insect species.

In the event that two B.t. Cry toxins compete for the same receptor, then if that receptor mutates in that insect so that one of the toxins no longer binds to that receptor and thus is no longer insecticidal against the insect, it might also be the case that the insect will also be resistant to the second toxin (which competitively bound to the same receptor). However, if two toxins bind to two different receptors, this could be an indication that the insect would not be simultaneously resistant to those two toxins.

Cry1Fa is useful in controlling many lepidopteran pests species including the European corn borer (ECB; Ostrinia nubilalis (Hübner)) and the fall armyworm (FAW; Spodoptera frugiperda), and is active against the sugarcane borer (SCB; Diatraea saccharalis).

The Cry1Fa protein, as produced in corn plants containing event TC1507, is responsible for an industry-leading insect resistance trait for FAW control. Cry1Fa is further deployed in the Herculex®, SmartStax™, and WideStrike™ products.

The ability to conduct (competitive or homologous) receptor binding studies using Cry1Fa protein has been limited because a common technique available for labeling proteins for detection in receptor binding assays tends to inactivate the insecticidal activity of the Cry1Fa protein.

Cry1Ab and Cry1Fa are insecticidal proteins currently used (separately) in transgenic corn to protect plants from a variety of insect pests. A key pest of corn that these proteins provide protection from is the European corn borer (ECB). US 2008/0311096 relates in part to the use of Cry1Ab to control a Cry1F-resistant ECB population.

### BRIEF SUMMARY OF THE INVENTION

The subject invention relates in part to the surprising discovery that Cry1Fa is very active against a sugarcane borer (SCB) population that is resistant to Cry1Ab. As one skilled in the art will recognize with the benefit of this disclosure, sugarcane plants producing Cry1Fa and Cry1Ab (including insecticidal portions thereof), will be useful in delaying or preventing the development of resistance by SCB to either of these insecticidal proteins alone.

### DETAILED DESCRIPTION OF THE INVENTION

The subject invention relates in part to the surprising discovery that Cry1Fa is very active against a sugarcane borer (SCB; Diatraea saccharalis) population that is resistant to Cry1Ab. Accordingly, the subject invention relates in part to the surprising discovery that Cry1Fa can be used in combination with, or “stacked” with, Cry1Ab in sugarcane to combat the development of resistance by SCB to either of these insecticidal proteins alone. Stated another way, the subject invention relates in part to the surprising discovery that a sugarcane borer population selected for resistance to Cry1Ab is not resistant to Cry1Fa; sugarcane borer that are resistant to Cry1Ab toxin are susceptible (i.e., are not cross-resistant) to Cry1Fa. Thus, the subject invention includes the use of Cry1Fa toxin in sugarcane to control populations of sugarcane borer that are resistant to Cry1Ab.

As one skilled in the art will recognize with the benefit of this disclosure, sugarcane plants expressing cry1Fa and cry1Ab (including insecticidal portions thereof), will be useful in delaying or preventing the development of resistance to either of these insecticidal proteins alone.

The subject invention includes the use of Cry1Fa and Cry1Ab to protect sugarcane from damage and yield loss caused by sugarcane borer or to sugarcane borer populations that have developed resistance to Cry1Ab.

The subject invention thus teaches an IRM stack to mitigate against the development of resistance by sugarcane borer to Cry1Ab and/or Cry1Fa.

Based in part on the data described herein, co-expressing cry1Fa and cry1Ab genes in sugarcane can produce a high dose IRM stack for controlling SCB. Other proteins can be added to this combination to add spectrum.

These data suggest that Cry1Fa would be effective in controlling SCB populations that have developed resistance to Cry1Ab. One deployment option would be to use these Cry proteins in geographies where Cry1Ab has become ineffective in controlling SCB due to the development of resistance. Another deployment option would be to use one or both of these Cry proteins in combination with Cry1Ab to mitigate the development of resistance in SCB to Cry1Ab.

Chimeric toxins of the subject invention comprise a full core N-terminal toxin portion of a B.t. toxin and, at some point past the end of the toxin portion, the protein has a transition to a heterologous protoxin sequence. The N-terminal toxin portion of a B.t. toxin is referered to herein as the “core” toxin. The transition to the heterologous protoxin segment can occur at approximately the toxin/protoxin junction or, in the alternative, a portion of the native protoxin (extending past the toxin portion) can be retained with the transition to the heterologous protoxin occurring downstream.

As an example, one chimeric toxin of the subject invention has the full core toxin portion of Cry1Ab (amino acids 1 to 601) and a heterologous protoxin (amino acids 602 to the C-terminus). In one preferred embodiment, the portion of a chimeric toxin comprising the protoxin is derived from a Cry1Ab protein toxin. As a second Example, a second chimeric toxin of the subject invention, has the full core toxin portion of Cry1Ca (amino acids 1 to 619) and a heterologous protoxin (amino acids 620 to the C-terminus). In a preferred embodiment, the portion of a chimeric toxin comprising the protoxin is derived from a Cry1Ab protein toxin. (The above can also be applied to Cry1Fa insecticidal proteins.) Unless otherwise specified, sequences can be obtained as described in US 2008/0311096.

A person skilled in this art will appreciate that B.t. toxins, even within a certain class such as cry1Fa or Cry1Ab, will vary to some extent in length and the precise location of the transition from toxin portion to protoxin portion. Typically, the cry1Fa toxins are about 1150 to about 1200 amino acids in length. The transition from toxin portion to protoxin portion will typically occur at between about 50% to about 60% of the full length toxin. The chimeric toxin of the subject invention will include the full expanse of this core N-terminal toxin portion. Thus, the chimeric toxin will comprise at least about 50% of the full length cry1Fa or Cry1Ab B.t. toxin. This will typically be at least about 590 amino acids. With regard to the protoxin portion, the full expanse of the cry1A(b) protoxin portion extends from the end of the toxin portion to the C-terminus of the molecule. It is the last about 100 to 150 amino acids of this portion which are most critical to include in the chimeric toxin of the subject invention.

Genes and toxins. The genes and toxins useful according to the subject invention include not only the full length sequences disclosed but also fragments of these sequences, variants, mutants, and fusion proteins which retain the characteristic pesticidal activity of the toxins specifically exemplified herein. As used herein, the terms “variants” or “variations” of genes refer to nucleotide sequences which encode the same toxins or which encode equivalent toxins having pesticidal activity. As used herein, the term “equivalent toxins” refers to toxins having the same or essentially the same biological activity against the target pests as the claimed toxins.

As used herein, the boundaries represent approximately 95% (Cry1Ab's and 1Fa's), 78% (Cry1A's and Cry1F's), and 45% (Cry1's) sequence identity, per “Revision of the Nomenclature for the Bacillus thuringiensis Pesticidal Crystal Proteins,” N. Crickmore, D. R. Zeigler, J. Feitelson, E. Schnepf, J. Van Rie, D. Lereclus, J. Baum, and D. H. Dean. Microbiology and Molecular Biology Reviews (1998) Vol 62: 807-813. These cut offs can also be applied to the core toxins only (for Cry1Ab and Cry1Fa toxins).

It should be apparent to a person skilled in this art that genes encoding active toxins can be identified and obtained through several means. The specific genes or gene portions exemplified herein may be obtained from the isolates deposited at a culture depository as described above. These genes, or portions or variants thereof, may also be constructed synthetically, for example, by use of a gene synthesizer. Variations of genes may be readily constructed using standard techniques for making point mutations. Also, fragments of these genes can be made using commercially available exonucleases or endonucleases according to standard procedures. For example, enzymes such as Bal31 or site-directed mutagenesis can be used to systematically cut off nucleotides from the ends of these genes. Also, genes which encode active fragments may be obtained using a variety of restriction enzymes. Proteases may be used to directly obtain active fragments of these toxins.

Fragments and equivalents which retain the pesticidal activity of the exemplified toxins would be within the scope of the subject invention. Also, because of the redundancy of the genetic code, a variety of different DNA sequences can encode the amino acid sequences disclosed herein. It is well within the skill of a person trained in the art to create these alternative DNA sequences encoding the same, or essentially the same, toxins. These variant DNA sequences are within the scope of the subject invention. As used herein, reference to “essentially the same” sequence refers to sequences which have amino acid substitutions, deletions, additions, or insertions which do not materially affect pesticidal activity. Fragments retaining pesticidal activity are also included in this definition.

A further method for identifying the gene-encoding toxins and gene portions useful according to the subject invention is through the use of oligonucleotide probes. These probes are detectable nucleotide sequences. These sequences may be detectable by virtue of an appropriate label or may be made inherently fluorescent as described in International Application No. WO93/16094. As is well known in the art, if the probe molecule and nucleic acid sample hybridize by forming a strong bond between the two molecules, it can be reasonably assumed that the probe and sample have substantial homology. Preferably, hybridization is conducted under stringent conditions by techniques well-known in the art, as described, for example, in Keller, G. H., M. M. Manak (1987) DNA Probes, Stockton Press, New York, N.Y., pp. 169-170. Some examples of salt concentrations and temperature combinations are as follows (in order of increasing stringency): 2×SSPE or SSC at room temperature; 1×SSPE or SSC at 42° C.; 0.1×SSPE or SSC at 42° C.; 0.1×SSPE or SSC at 65° C. Detection of the probe provides a means for determining in a known manner whether hybridization has occurred. Such a probe analysis provides a rapid method for identifying toxin-encoding genes of the subject invention. The nucleotide segments which are used as probes according to the invention can be synthesized using DNA synthesizer and standard procedures. These nucleotide sequences can also be used as PCR primers to amplify genes of the subject invention.

Certain toxins of the subject invention have been specifically exemplified herein. Since these toxins are merely exemplary of the toxins of the subject invention, it should be readily apparent that the subject invention comprises variant or equivalent toxins (and nucleotide sequences coding for equivalent toxins) having the same or similar pesticidal activity of the exemplified toxin. Equivalent toxins will have amino acid homology with an exemplified toxin. This amino acid homology will typically be greater than 75%, preferably be greater than 90%, and most preferably be greater than 95%. The amino acid homology will be highest in critical regions of the toxin which account for biological activity or are involved in the determination of three-dimensional configuration which ultimately is responsible for the biological activity. In this regard, certain amino acid substitutions are acceptable and can be expected if these substitutions are in regions which are not critical to activity or are conservative amino acid substitutions which do not affect the three-dimensional configuration of the molecule. For example, amino acids may be placed in the following classes: non-polar, uncharged polar, basic, and acidic. Conservative substitutions whereby an amino acid of one class is replaced with another amino acid of the same type fall within the scope of the subject invention so long as the substitution does not materially alter the biological activity of the compound. Table 1 provides a listing of examples of amino acids belonging to each class.

TABLE 1

| Class of Amino Acid | Examples of Amino Acids |
|---|---|
| Nonpolar | Ala, Val, Leu, Ile, Pro, Met, Phe, Trp |
| Uncharged Polar | Gly, Ser, Thr, Cys, Tyr, Asn, Gln |
| Acidic | Asp, Glu |
| Basic | Lys, Arg, His |

In some instances, non-conservative substitutions can also be made. The critical factor is that these substitutions must not significantly detract from the biological activity of the toxin.

Recombinant hosts. The genes encoding the toxins of the subject invention can be introduced into a wide variety of microbial or plant hosts. Expression of the toxin gene results, directly or indirectly, in the intracellular production and maintenance of the pesticide. Conjugal transfer and recombinant transfer can be used to create a B.t. strain that expresses both toxins of the subject invention. Other host organisms may also be transformed with one or both of the toxin genes then used to accomplish the synergistic effect. With suitable microbial hosts, e.g., Pseudomonas, the microbes can be applied to the situs of the pest, where they will proliferate and be ingested. The result is control of the pest. Alternatively, the microbe hosting the toxin gene can be treated under conditions that prolong the activity of the toxin and stabilize the cell. The treated cell, which retains the toxic activity, then can be applied to the environment of the target pest.

Where the B.t. toxin gene is introduced via a suitable vector into a microbial host, and said host is applied to the environment in a living state, it is essential that certain host microbes be used. Microorganism hosts are selected which are known to occupy the “phytosphere” (phylloplane, phyllosphere, rhizosphere, and/or rhizoplane) of one or more crops of interest. These microorganisms are selected so as to be capable of successfully competing in the particular environment (crop and other insect habitats) with the wild-type microorganisms, provide for stable maintenance and expression of the gene expressing the polypeptide pesticide, and, desirably, provide for improved protection of the pesticide from environmental degradation and inactivation.

A large number of microorganisms are known to inhabit the phylloplane (the surface of the plant leaves) and/or the rhizosphere (the soil surrounding plant roots) of a wide variety of important crops. These microorganisms include bacteria, algae, and fungi. Of particular interest are microorganisms, such as bacteria, e.g., genera Pseudomonas, Erwinia, Serratia, Klebsiella, Xanthomonas, Streptomyces, Rhizobium, Rhodopseudomonas, Methylophilius, Agrobactenum, Acetobacter, Lactobacillus, Arthrobacter, Azotobacter, Leuconostoc, and Alcaligenes; fungi, particularly yeast, e.g., genera Saccharomyces, Cryptococcus, Kluyveromyces, Sporobolomyces, Rhodotorula, and Aureobasidium. Of particular interest are such phytosphere bacterial species as Pseudomonas syringae, Pseudomonas fluorescens, Serratia marcescens, Acetobacter xylinum, Agrobactenium tumefaciens, Rhodopseudomonas spheroides, Xanthomonas campestris, Rhizobium melioti, Alcaligenes entrophus, and Azotobacter vinlandii; and phytosphere yeast species such as Rhodotorula rubra, R. glutinis, R. marina, R. aurantiaca, Cryptococcus albidus, C. diffluens, C. laurentii, Saccharomyces rosei, S. pretoriensis, S. cerevisiae, Sporobolomyces roseus, S. odorus, Kluyveromyces veronae, and Aureobasidium pollulans. Of particular interest are the pigmented microorganisms.

A wide variety of ways are available for introducing a B.t. gene encoding a toxin into a microorganism host under conditions which allow for stable maintenance and expression of the gene. These methods are well known to those skilled in the art and are described, for example, in U.S. Pat. No. 5,135,867, which is incorporated herein by reference.

Treatment of cells. Bacillus thuringiensis or recombinant cells expressing the B.t. toxins can be treated to prolong the toxin activity and stabilize the cell. The pesticide microcapsule that is formed comprises the B.t. toxin or toxins within a cellular structure that has been stabilized and will protect the toxin when the microcapsule is applied to the environment of the target pest. Suitable host cells may include either prokaryotes or eukaryotes, normally being limited to those cells which do not produce substances toxic to higher organisms, such as mammals. However, organisms which produce substances toxic to higher organisms could be used, where the toxic substances are unstable or the level of application sufficiently low as to avoid any possibility of toxicity to a mammalian host. As hosts, of particular interest will be the prokaryotes and the lower eukaryotes, such as fungi.

The cell will usually be intact and be substantially in the proliferative form when treated, rather than in a spore form, although in some instances spores may be employed.

Treatment of the microbial cell, e.g., a microbe containing the B.t. toxin gene or genes, can be by chemical or physical means, or by a combination of chemical and/or physical means, so long as the technique does not deleteriously affect the properties of the toxin, nor diminish the cellular capability of protecting the toxin. Examples of chemical reagents are halogenating agents, particularly halogens of atomic no. 17-80. More particularly, iodine can be used under mild conditions and for sufficient time to achieve the desired results. Other suitable techniques include treatment with aldehydes, such as glutaraldehyde; anti-infectives, such as zephiran chloride and cetylpyridinium chloride; alcohols, such as isopropyl and ethanol; various histologic fixatives, such as Lugol iodine, Bouin's fixative, various acids and Helly's fixative (See: Humason, Gretchen L., Animal Tissue Techniques, W. H. Freeman and Company, 1967); or a combination of physical (heat) and chemical agents that preserve and prolong the activity of the toxin produced in the cell when the cell is administered to the host environment. Examples of physical means are short wavelength radiation such as gamma-radiation and X-radiation, freezing, UV irradiation, lyophilization, and the like. Methods for treatment of microbial cells are disclosed in U.S. Pat. Nos. 4,695,455 and 4,695,462, which are incorporated herein by reference.

The cells generally will have enhanced structural stability which will enhance resistance to environmental conditions. Where the pesticide is in a proform, the method of cell treatment should be selected so as not to inhibit processing of the proform to the mature form of the pesticide by the target pest pathogen. For example, formaldehyde will crosslink proteins and could inhibit processing of the proform of a polypeptide pesticide. The method of treatment should retain at least a substantial portion of the bio-availability or bioactivity of the toxin.

Characteristics of particular interest in selecting a host cell for purposes of production include ease of introducing the B.t. gene or genes into the host, availability of expression systems, efficiency of expression, stability of the pesticide in the host, and the presence of auxiliary genetic capabilities. Characteristics of interest for use as a pesticide microcapsule include protective qualities for the pesticide, such as thick cell walls, pigmentation, and intracellular packaging or formation of inclusion bodies; survival in aqueous environments; lack of mammalian toxicity; attractiveness to pests for ingestion; ease of killing and fixing without damage to the toxin; and the like. Other considerations include ease of formulation and handling, economics, storage stability, and the like.

Growth of cells. The cellular host containing the B.t. insecticidal gene or genes may be grown in any convenient nutrient medium, where the DNA construct provides a selective advantage, providing for a selective medium so that substantially all or all of the cells retain the B.t. gene. These cells may then be harvested in accordance with conventional ways. Alternatively, the cells can be treated prior to harvesting.

The B.t. cells producing the toxins of the invention can be cultured using standard art media and fermentation techniques. Upon completion of the fermentation cycle the bacteria can be harvested by first separating the B.t. spores and crystals from the fermentation broth by means well known in the art. The recovered B.t. spores and crystals can be formulated into a wettable powder, liquid concentrate, granules or other formulations by the addition of surfactants, dispersants, inert carriers, and other components to facilitate handling and application for particular target pests. These formulations and application procedures are all well known in the art.

Formulations. Formulated bait granules containing an attractant and spores, crystals, and toxins of the B.t. isolates, or recombinant microbes comprising the genes obtainable from the B.t. isolates disclosed herein, can be applied to the soil. Formulated product can also be applied as a seed-coating or root treatment or total plant treatment at later stages of the crop cycle. Plant and soil treatments of B.t. cells may be employed as wettable powders, granules or dusts, by mixing with various inert materials, such as inorganic minerals (phyllosilicates, carbonates, sulfates, phosphates, and the like) or botanical materials (powdered corncobs, rice hulls, walnut shells, and the like). The formulations may include spreader-sticker adjuvants, stabilizing agents, other pesticidal additives, or surfactants. Liquid formulations may be aqueous-based or non-aqueous and employed as foams, gels, suspensions, emulsifiable concentrates, or the like. The ingredients may include rheological agents, surfactants, emulsifiers, dispersants, or polymers.

As would be appreciated by a person skilled in the art, the pesticidal concentration will vary widely depending upon the nature of the particular formulation, particularly whether it is a concentrate or to be used directly. The pesticide will be present in at least 1% by weight and may be 100% by weight. The dry formulations will have from about 1-95% by weight of the pesticide while the liquid formulations will generally be from about 1-60% by weight of the solids in the liquid phase. The formulations will generally have from about 10.sup.2 to about 10.sup.4 cells/mg. These formulations will be administered at about 50 mg (liquid or dry) to 1 kg or more per hectare.

The formulations can be applied to the environment of the lepidopteran pest, e.g., foliage or soil, by spraying, dusting, sprinkling, or the like.

Plant transformation. A preferred recombinant host for production of the insecticidal proteins of the subject invention is a transformed plant. Genes encoding Bt toxin proteins, as disclosed herein, can be inserted into plant cells using a variety of techniques which are well known in the art. For example, a large number of cloning vectors comprising a replication system in Escherichia coli and a marker that permits selection of the transformed cells are available for preparation for the insertion of foreign genes into higher plants. The vectors comprise, for example, pBR322, pUC series, M13mp series, pACYC184, inter alia. Accordingly, the DNA fragment having the sequence encoding the Bt toxin protein can be inserted into the vector at a suitable restriction site. The resulting plasmid is used for transformation into E. coli. The E. coli cells are cultivated in a suitable nutrient medium, then harvested and lysed. The plasmid is recovered. Sequence analysis, restriction analysis, electrophoresis, and other biochemical-molecular biological methods are generally carried out as methods of analysis. After each manipulation, the DNA sequence used can be cleaved and joined to the next DNA sequence. Each plasmid sequence can be cloned in the same or other plasmids. Depending on the method of inserting desired genes into the plant, other DNA sequences may be necessary. If, for example, the Ti or Ri plasmid is used for the transformation of the plant cell, then at least the right border, but often the right and the left border of the Ti or Ri plasmid T-DNA, has to be joined as the flanking region of the genes to be inserted. The use of T-DNA for the transformation of plant cells has been intensively researched and sufficiently described in EP 120 516, Lee and Gelvin (2008), Hoekema (1985), Fraley et al., (1986), and An et al., (1985), and is well established in the art.

Once the inserted DNA has been integrated in the plant genome, it is relatively stable. The transformation vector normally contains a selectable marker that confers on the transformed plant cells resistance to a biocide or an antibiotic, such as Bialaphos, Kanamycin, G418, Bleomycin, or Hygromycin, inter alia. The individually employed marker should accordingly permit the selection of transformed cells rather than cells that do not contain the inserted DNA.

A large number of techniques is available for inserting DNA into a plant host cell. Those techniques include transformation with T-DNA using Agrobacterium tumefaciens or Agrobacterium rhizogenes as transformation agent, fusion, injection, biolistics (microparticle bombardment), or electroporation as well as other possible methods. If Agrobacteria are used for the transformation, the DNA to be inserted has to be cloned into special plasmids, namely either into an intermediate vector or into a binary vector. The intermediate vectors can be integrated into the Ti or Ri plasmid by homologous recombination owing to sequences that are homologous to sequences in the T-DNA. The Ti or Ri plasmid also comprises the vir region necessary for the transfer of the T-DNA. Intermediate vectors cannot replicate themselves in Agrobacteria. The intermediate vector can be transferred into Agrobacterium tumefaciens by means of a helper plasmid (conjugation). Binary vectors can replicate themselves both in E. coli and in Agrobacteria. They comprise a selection marker gene and a linker or polylinker which are framed by the Right and Left T-DNA border regions. They can be transformed directly into Agrobacteria (Holsters et al., 1978). The Agrobacterium used as host cell is to comprise a plasmid carrying a vir region. The vir region is necessary for the transfer of the T-DNA into the plant cell. Additional T-DNA may be contained. The bacterium so transformed is used for the transformation of plant cells. Plant explants can advantageously be cultivated with Agrobacterium tumefaciens or Agrobacterium rhizogenes for the transfer of the DNA into the plant cell. Whole plants can then be regenerated from the infected plant material (for example, pieces of leaf, segments of stalk, roots, but also protoplasts or suspension-cultivated cells) in a suitable medium, which may contain antibiotics or biocides for selection. The plants so obtained can then be tested for the presence of the inserted DNA. No special demands are made of the plasmids in the case of injection and electroporation. It is possible to use ordinary plasmids, such as, for example, pUC derivatives.

The transformed cells grow inside the plants in the usual manner. They can form germ cells and transmit the transformed trait(s) to progeny plants. Such plants can be grown in the normal manner and crossed with plants that have the same transformed hereditary factors or other hereditary factors. The resulting hybrid individuals have the corresponding phenotypic properties.

In a preferred embodiment of the subject invention, plants will be transformed with genes wherein the codon usage has been optimized for plants. See, for example, U.S. Pat. No. 5,380,831, which is hereby incorporated by reference. While some truncated toxins are exemplified herein, it is well-known in the Bt art that 130 kDa-type (full-length) toxins have an N-terminal half that is the core toxin, and a C-terminal half that is the protoxin “tail.” Thus, appropriate “tails” can be used with truncated/core toxins of the subject invention. See e.g. U.S. Pat. No. 6,218,188 and U.S. Pat. No. 6,673,990. In addition, methods for creating synthetic Bt genes for use in plants are known in the art (Stewart and Burgin, 2007). One non-limiting example of a preferred transformed plant is a fertile maize plant comprising a plant expressible gene encoding a Cry1Fa protein, and further comprising a second plant expressible gene encoding a Cry1Ab protein.

Transfer (or introgression) of the Cry1Ab and Cry1Fa trait(s) into inbred maize lines can be achieved by recurrent selection breeding, for example by backcrossing. In this case, a desired recurrent parent is first crossed to a donor inbred (the non-recurrent parent) that carries the appropriate gene(s) for the Cry1Ab and Cry1Fa traits. The progeny of this cross is then mated back to the recurrent parent followed by selection in the resultant progeny for the desired trait(s) to be transferred from the non-recurrent parent. After three, preferably four, more preferably five or more generations of backcrosses with the recurrent parent with selection for the desired trait(s), the progeny will be heterozygous for loci controlling the trait(s) being transferred, but will be like the recurrent parent for most or almost all other genes (see, for example, Poehlman & Sleper (1995) Breeding Field Crops, 4th Ed., 172-175; Fehr (1987) Principles of Cultivar Development, Vol. 1: Theory and Technique, 360-376).

Insect Resistance Management (IRM) Strategies. Roush et al., for example, outlines two-toxin strategies, also called “pyramiding” or “stacking,” for management of insecticidal transgenic crops. (The Royal Society. Phil. Trans. R. Soc. Lond. B. (1998) 353, 1777-1786). On their website, the U.S. Environmental Protection Agency provides the following guidance, for providing non-transgenic refuges (a block of non-Bt crops/corn) for use with transgenic crops.
(epa.gov/oppbppd1/biopesticides/pips/bt_corn_refuge_{—}2006.htm)
   The specific structured requirements for corn borer-protected Bt (Cry1Ab and Cry1F) corn products are as follows:
      Structured refuges: 20% non-Lepidopteran Bt corn refuge in Corn Belt; 50% non-Lepidopteran Bt refuge in Cotton Belt
         Blocks
            1. Internal (i.e., within the Bt field)
            2. External (i.e., separate fields within ½ mile (¼ mile if possible) of the Bt field to maximize random mating)
         In-field Strips
            Strips must be at least 4 rows wide (preferably 6 rows) to reduce the effects of larval movement
The National Corn Growers Association, on their website (ncga.com/insect-resistance-management-fact-sheet-bt-corn), also provides similar guidance regarding the requirements. For example:
   Requirements of the Corn Borer IRM:
      Plant at least 20% of your corn acres to refuge hybrids
      In cotton producing regions, refuge must be 50%
      Must be planted within ½ mile of the refuge hybrids
      Refuge can be planted as strips within the Bt field; the refuge strips must be at least 4 rows wide
      Refuge may be treated with conventional pesticides only if economic thresholds are reached for target insect
      Bt-based sprayable insecticides cannot be used on the refuge corn
      Appropriate refuge must be planted on every farm with Bt corn
As stated by Roush et al. (on pages 1780 and 1784 right column, for example), stacking or pyramiding can allow for use of a smaller refuge. Roush suggests approximately 10% refuge for a successful stack, compared to (and down) from about 30-40%.

Any of the above percentages (such as those for 1F/1Ab), or similar refuge ratios, can be used for the subject double or triple stacks or pyramids in sugarcane.

There are various ways of providing the refuge, including various geometric planting patterns in the fields (as mentioned above), to in-bag seed mixtures, as discussed further by Roush et al. (supra), and U.S. Pat. No. 6,551,962.

All patents, patent applications, provisional applications, and publications referred to or cited herein are incorporated by reference in their entirety to the extent they are not inconsistent with the explicit teachings of this specification.

The following examples illustrate the invention. The examples should not be construed as limiting.

### EXAMPLES

### Example 1

### Summary—Response of a Cry1Ab-Susceptible and -Resistant Sugarcane Borer to Cry1Fa Bacillus thuringiensis Cry Protein

Cry1Fa protein demonstrated insecticidal activity against both Bt-susceptible (Bt-SS) and Bt-resistant (Bt-RR) strains of the sugarcane borer, Diatraea saccharalis. The Bt-RR strain of D. saccharalis demonstrated a 142-fold resistance to trypsin-activated Cry1Ab protein. This Bt-resistant strain of D. saccharalis showed some cross-resistance to Cry1Fa, but the resistance ratios were reduced significantly (4-fold). The results suggest that Cry1Fa can be effective for managing Cry1Ab resistance in D. saccharalis and other corn borer species.

### Example 2

### Materials and Methods

Bacillus thuringiensis Cry Proteins

Purified trypsin-activated Bacillus thuringiensis (Bt) Cry1Ab protein was obtained from Dr. Marianne Purtai-Carey, Department of Biochemistry, Case Western Reserve University, Cleveland, Ohio. Cry1Fa was provided by Dow AgroSciences Company (Indianapolis, Ind.) in a buffer solution. The Cry1Ab was lyophilized with a purity level of 99.9%.

Insect Sources

A Bt-susceptible strain (Bt-SS) of D. saccharalis was established using larvae collected from corn fields near Winnsboro in Northeast Louisiana during 2004. A Bt-resistant strain (Bt-RR) of D. saccharalis was developed from a single iso-line family using an F₂ screen. These Bt-resistant insects completed larval development on commercial Cry1Ab corn hybrids and demonstrated a significant resistance level to purified trypsin-activated Cry1Ab toxin. During confirmation of Bt resistance, individuals of the Bt-resistant strain were backcrossed with those of the Bt-susceptible strain and re-selected for resistance with Cry1Ab corn leaf tissue in the F₂ generation of the backcross.

Insect Bioassays

Larval susceptibility of the Bt-SS and Bt-RR strains of D. saccharalis to Cry1Ab and Cry1Fa was determined using diet incorporation procedures. In each bioassay, 6 or 7 Cry protein concentrations were used. The range of Bt concentrations was from 0.03125 to 32 μg/g for assaying Cry1Ab protein, and from 0.03125 to 128 for evaluating Cry1Fa. Cry protein solutions were prepared by mixing Bt proteins with appropriate amount of distilled water for assaying Cry1Ab or the buffer for examining Cry1Fa. The Bt solutions were then mixed with a meridic diet just prior to dispensing the diet into individual cells of 128-cell trays (Bio-Ba-128, C-D International, Pitman, N.J.). In the bioassay, approximately 0.7 ml of treated diet was placed into each cell using 10-ml syringes (Becton, Dickinson and Company, Franklin Lakes, N.J.). Diet treated with distilled water (blank control) or buffer only was used as control treatments. One neonate (<24 h) of D. saccharalis was released on the diet surface in each cell. After larval inoculation, cells were covered with vented lids (C-D International, Pitman, N.J.). The bioassay trays were placed in an environmental chamber maintained at 28° C., 50% RH, and a 16:8 (L:D) h photoperiod. Larval mortality, larval weight, and number of surviving larvae that did not demonstrate weight gains (<0.1 mg per larva) were recorded on the 7^{th} day after inoculation. Each combination of insect strain by Cry protein concentration was replicated four times with 16 to 32 larvae in each replicate.

Data Analysis

Larval mortality criteria were measured as ‘practical’ mortality, which considered both the actual dead larvae and the surviving larvae that did not show a significant gain in body weight (<0.1 mg per larva) as morbid or non-feeding insects. The practical mortality of D. saccharalis in a treatment was calculated using the equation: Practical mortality (%)=100×[number of dead larvae+number of surviving larvae that did not show a significant gain in body weight (<0.1 mg per larva)]/total number of insects tested. The ‘practical’ mortality (hereafter simplified as mortality) of each D. saccharalis strain was corrected for larval mortality on non-treated control diet for analyzing Cry1Ab or the buffer only-treated diet for assessing Cry1Fa. Corrected dose/mortality data then were subjected to probit analysis for determining Cry protein concentrations that caused 50% (LC₅₀) mortality value and the corresponding 95% confidence intervals (CI). The treatments used in the probit analysis included the highest concentration that produced zero mortality, the lowest concentration that resulted in 100% mortality, and all results between those extremes. Resistance ratios were calculated by dividing the LC₅₀ value of the Bt-RR strain by that of the Bt-SS insects. A lethal dose ratio test was used to determine if the resistance ratios were significant at α=0.05 level. A two-way ANOVA also was used to analyze the mortality data, followed by the LSMEANS test at the α=0.05 level to determine treatment differences.

Larval growth inhibition of D. saccharalis on a Cry1Ab protein diet was calculated using the formula: larval growth inhibition (%)=100×(body weight of larvae feeding on non-treated control diet−body weight of larvae feeding on Bt diet)/(body weight of larvae feeding on non-treated control diet), whereas, for analyzing Cry1Fa, it was calculated using the formula: larval growth inhibition (%)=100×(body weight of larvae feeding on buffer only treated control diet−body weight of larvae feeding on Bt diet)/(body weight of larvae feeding on buffer only treated control diet). A 100% of larval growth inhibition was assigned to a replication if there were no larvae that had significant weight gain (<0.1 mg/larva). The growth inhibition data were analyzed using a two-way ANOVA with insect strain and Cry protein concentration as the two main factors. LSMEANS tests were used to determine treatment differences at the α=0.05 level. Non-transformed data are presented in the figures and tables.

### Example 3

### Results

Larval Mortality of Bt-SS and Bt-RR Strains of D. saccharalis on Cry Protein-Treated Diet.

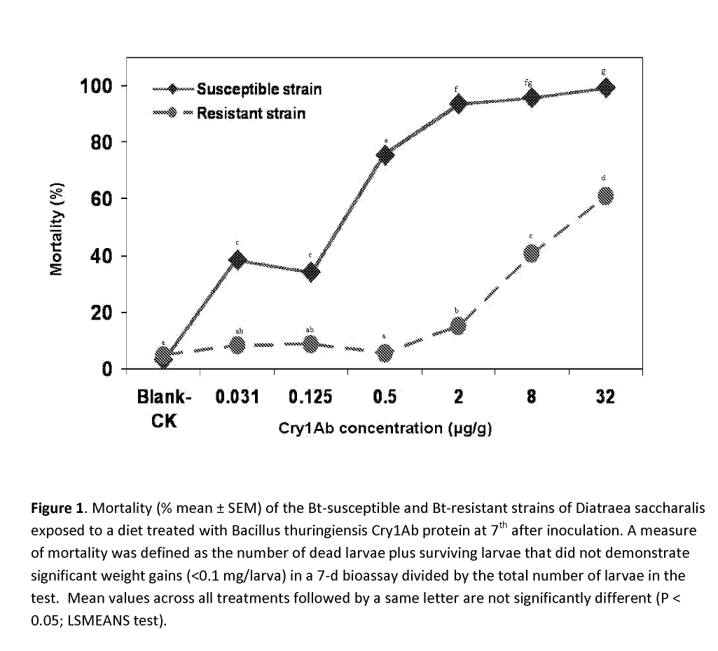

Cry1Ab protein (FIG. 1): Cry1Ab protein concentration had a significant effect on larval mortality of D. saccharalis for both Bt-SS and Bt-RR strains (F=90.67; df=6, 42; P<0.0001) (FIG. 1). Larval mortality increased as Cry1Ab concentration increased. Significant levels of larval mortality of the Bt-SS strain was observed at 0.031 μg/g or higher and the mortality reached near 100% at 32 μg/g. For the Bt-RR strain, significant mortality occurred at 2 μg/g and reached 61% at 32 μg/g. Considerable differences in larval mortality was observed between the two insect strains (F=346.73, df.=1, 42, P<0.0001). Larval mortality of Bt-RR strain was significantly (P<0.05) lower than that of Bt-SS insects at all Cry1Ab concentrations examined. The interaction of insect strain and concentration was also significant (F=18.82; df=6, 42; P<0.0001). Larval mortality of Bt-RR strain increased slower than that of the Bt-SS strain as Cry1Ab concentration increased.

The calculated LC₅₀ values based on larval mortality for the Bt-SS and Bt-RR strains were 0.13 and 18.46 μg/g, respectively (Table 1). The 142-fold difference in the LC₅₀s between the two strains was significant (P<0.05) based on the lethal dose ratio test.

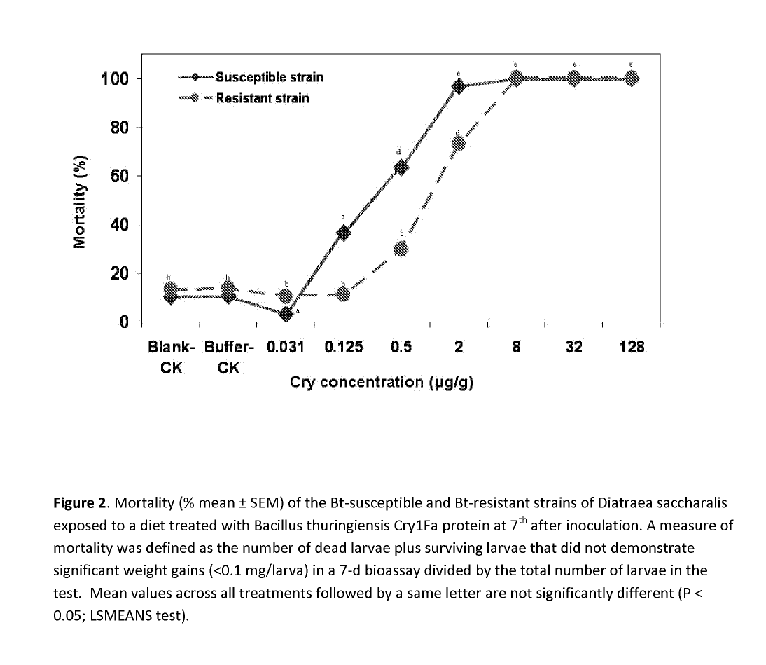

Cry1Fa protein (FIG. 2): Cry1Fa protein demonstrated insecticidal activity and only some cross resistance. Cry protein concentration had a significant effect on larval mortality of D. saccharalis for both Bt-SS and Bt-RR strains (F=251.78; df=8, 54; P<0.0001). Significant levels of larval mortality was observed at 0.125 μg/g for Bt-SS and 0.5 μg/g for Bt-RR strains and reached 100% at 8 μg/g for both strains. Differences in larval mortality also was significant between the two insect strains (F=11.82; df.=1, 54; P=0.0011). Bt-RR strain had a significantly (P<0.05) lower mortality at 0.125, 0.5, and 2 μg/g than Bt-SS strain. The interaction of insect strain and concentration was also significant (F=8.61; df=8, 54; P<0.0001). In general, larval mortality of Bt-RR strain at Cry protein concentrations of <8 μg/g increased slower than that of the Bt-SS strain.

The calculated LC₅₀ values based on larval mortality for the Bt-SS and Bt-RR strains were 0.29 and 1.15 μg/g, respectively (Table 1). The 4-fold difference in the LC₅₀s between the two strains was statistically significant (P<0.05) based on the lethal dose ratio test.

Larval Growth Inhibition of D. saccharalis on Cry Protein-Treated Diet

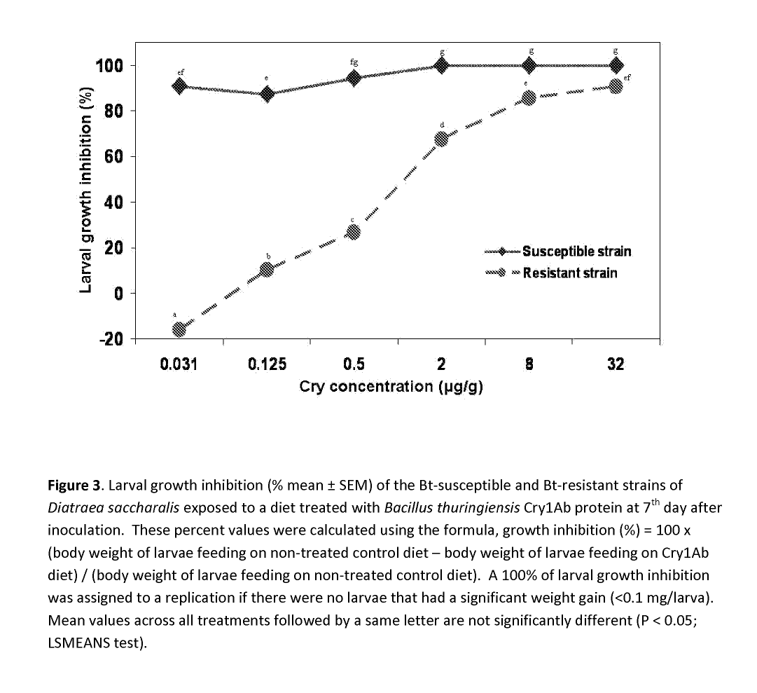

Cry1Ab protein (FIG. 3): Larval growth inhibition of the Bt-SS and the Bt-RR strains of D. saccharalis on Cry1Ab treated diet was significantly different among concentrations (F=175.07; df=5, 36; P<0.0001). Growth of the Bt-SS and Bt-RR larvae decreased as Cry1Ab concentrations increased. The effect of insect strain on growth inhibition was significantly different between Bt-SS and Bt-RR strains (F=1182.51; df=1, 36; P<0.0001). Larval growth inhibition of Bt-SS strain was significantly greater than that of Bt-RR strain across all Bt concentrations tested. At the concentration of 0.031 μg/g, the lowest concentration tested, Bt-RR did not show any growth inhibition, but Bt-SS larvae had a >90% growth inhibition compared to the control. At 0.5 μg/g, Bt-RR demonstrated a 27% growth inhibition, whereas growth of Bt-SS larvae was nearly completely stopped. The interaction of insect strain and Bt concentration also was significant (F=110.72; df=5, 36; P<0.0001). Larval growth inhibition for the Bt-RR strain increased slower as Cry1Ab concentrations increased than that of the Bt SS strain

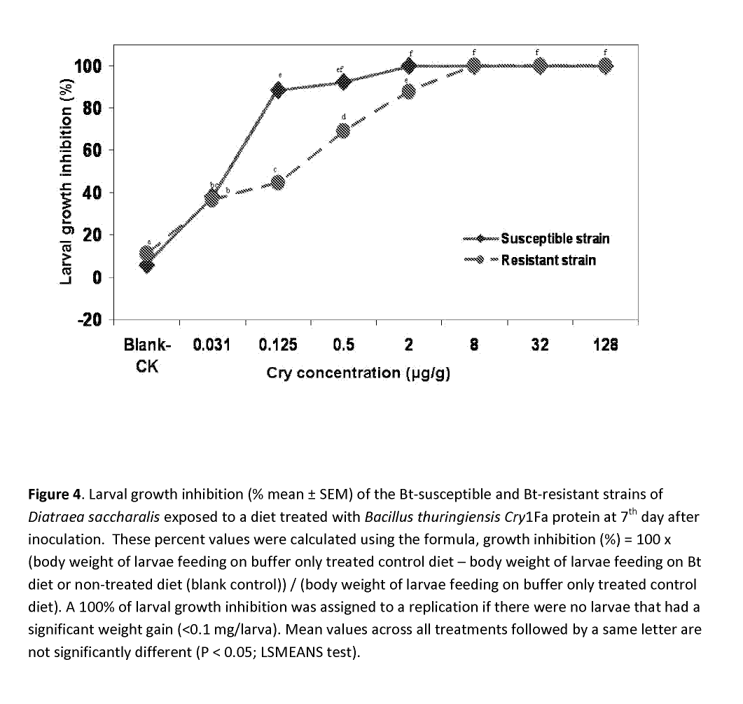

Cry1Fa protein (FIG. 4): Larval growth inhibition of the Bt-SS and the Bt-RR strains of D. saccharalis on Cry1Fa protein-treated diet was significantly different among concentrations (F=301.69; df=7, 48; P<0.0001). Growth inhibition of the Bt-SS was significantly greater (P<0.05) than that of Bt-RR larvae at the concentrations of 0.125, 0.5, and 2 μg/g. The effect of insect strain on growth inhibition was significantly different between the two insect strains (F=45.88; df=1, 48; P<0.0001) and the interaction of insect strain and Bt concentration also was significant (F=18.38; df=7, 48; P<0.0001). Growth inhibition of Bt-SS strain increased faster than that of Bt-RR strain. Significant larval growth inhibition of both insect strains was observed at 0.03125 μg/g. The growth of Bt-SS strain was completely inhibited at 2 μg/g, while it occurred at 8 μg/g for Bt-RR strain.

### REFERENCES

Finney, D. J. 1971. Probit analysis. Cambridge University Press, England.
Hua, G., L. Masson, J. L. Jurat-Fuentes, G. Schwab, and M. J. Adang. Binding analyses of Bacillus thuringiensis Cry d-endotoxins using brush border membrane vesicles of Ostrinia nubilalis. Applied and Environmental Microbiology 67[2], 872-879. 2001.
LeOra Software. 1987. POLO-PC. A user's guide to probit and logit analysis. Berkeley, Calif.
McGaughey, W. H., F. Gould, and W. Gelernter. Bt resistance management. Nature Biotechnology 16[2], 144-146. 1998
Marçon, P. R. G. C., L. J. Young, K. Steffey, and B. D. Siegfried. 1999. Baseline susceptibility of the European corn borer, Ostrinia nubilalis (Hübner) (Lepidoptera: Pyralidae) to Bacillus thuringiensis toxins. J. Econ. Entomol. 92 (2): 280-285.
Robertson, L. J. and H. K. Preisler. 1992. Pesticide bioassays with arthropods. CRC Press, Boca Ranton, Fla.
SAS Institute Inc. 1988. SAS procedures guide, Release 6.03 edition. SAS Institute Inc, Cary, N.C.
Stone, B. F. 1968. A formula for determining degree of dominance in cases of monofactorial inheritance of resistance to chemicals. Bull. WHO 38:325-329.
Van Mellaert, H., J. Botterman, J. Van Rie, and H. Joos. Transgenic plants for the prevention of development of insects resistant to Bacillus thuringiensis toxins. (Plant Genetic Systems N.V., Belg. 89-401499[400246], 57-19901205. EP. May 31, 1989

We claim:

1. A sugarcane plant comprising DNA encoding a Cry1Fa insecticidal protein and DNA encoding a Cry1Ab insecticidal protein.

2. The sugarcane plant of claim 1 wherein DNA encoding a Cry1Fa core toxin-containing protein and DNA encoding a Cry1Ab core toxin-containing protein have been introgressed into said sugarcane plant.

3. A part of a plant of claim 1.

4. A cutting or clonal propagate of a plant of claim 1.

5. A field of plants comprising non-Bt refuge plants and a plurality of sugarcane plants of claim 1, wherein said refuge plants comprise less than 40% of all the plants in said field.

6. The field of plants of claim 5, wherein said refuge plants comprise less than 30% of all the crop plants in said field.

7. The field of plants of claim 5, wherein said refuge plants comprise less than 20% of all the crop plants in said field.

8. The field of plants of claim 5, wherein said refuge plants comprise less than 10% of all the crop plants in said field.

9. The field of plants of claim 5, wherein said refuge plants comprise less than 5% of all the crop plants in said field.

10. The field of plants of claim 5, wherein said refuge plants are in blocks or strips.

11. The field of plants of claim 5, wherein said sugarcane plants occupy more than 10 acres.

12. The sugarcane plant of claim 1, wherein said Cry1Fa protein is at least 99% identical with SEQ ID NO:1, and said Cry1Ab protein is at least 99% identical with SEQ ID NO:2.
